(19) United States
(12) Patent Application Publication
(10) Pub. No.: US 2005/0049518 A1
(43) Pub. Date: Mar. 3, 2005

(54) Determining progresterone cycles in livestock

(76) Inventor: Andre Johan Nel, Pretoria (ZA)

Correspondence Address: MERCHANT & GOULD PC, P.O. BOX 2903, MINNEAPOLIS, MN, 55402-0903, US

(21) Appl. No.: 10/495,447

(22) PCT Filed: Nov. 11, 2002
(86) PCT No.: PCT/ZA02/00171

(30) Foreign Application Priority Data
Nov. 12, 2001 (ZA) 2001//9289

(51) Int. Cl. A61B 5/05

(52) U.S. Cl. 600/547; 600/551

(57) ABSTRACT

A method of determining hormone activity in a mammal includes the step of measuring muscle tissue impedance of the mammal. The hormone activity can be reproductive hormone activity of a female mammal.

### TECHNICAL FIELD OF THE INVENTION

This invention relates to a method for determining the hormone activity of mammalian livestock and a practical method of monitoring the progress of a female mammal's reproductive hormone or progesterone cycle. The invention also relates to a method and apparatus for measuring, monitoring and determining the reproductive hormone or progesterone cycle of livestock. The invention further relates to a system for monitoring and visually displaying the reproductive hormone cycle of livestock.

### BACKGROUND OF THE INVENTION

The applicant is aware that reproductive hormone activity effects the electrochemical properties of cells, such as the ion transport mechanisms of cells, particularly that of muscle cells. The closer the muscle tissue is to the uterus, the stronger the effect. The strongest effect occurs in the uterus muscle tissue. The applicant has found that the effect on the electrochemistry or ion transport mechanism in cells, in turn, has an effect on the capacitance and therefore on the impedance of tissue comprising the cells. Muscle tissue impedance is therefore influenced by the reproductive hormonal activity in the mammal, which follows a pattern over the course of the progesterone cycle. It is believed that the reproductive hormone progesterone conditions the muscle cells of the womb for delivery by creating low impedance paths for nerve pulses.

The monitoring of the reproductive hormone or progesterone cycle in livestock enables a person to monitor whether an individual mammal's reproductive hormone cycle is synchronised with that of a group of selected individuals of livestock. It further enables a person to determine whether an mammal's reproductive hormone cycle is abnormal or irregular, whether an mammal is ready for conception or insemination, whether an mammal is pregnant and further to obtain additional data relating to livestock reproduction.

### PRIOR ART DISCUSSION

Scott et al., U.S. Pat. No. 4,224,949, disclose a bovine vaginal probe capable of measuring electrical resistance of the vaginal mucus in a bovine vaginal tract by applying a voltage across poles of an electrode on the probe, to detect changes in electrical resistance indicative of oestrus.

Hofmeister, U.S. Pat. No. 4,577,640, discloses an apparatus for detecting and monitoring natural bio-electrical activity generated in the uterus and characterised by an electrode carrier in the form of a cup shaped to conform to the patients cervix. This patent also discloses a method of measuring uterine electrical activity that comprises monitoring and recording the electrical potential along at least two discrete current paths simultaneously with reference to a common time base.

Garfield et al., U.S. Pat. No. 5,522,877, discloses a method and apparatus for detecting gap junctions for diagnosing labour in pregnant woman by stimulating the nerves in the tissue with signals which do not stimulate the cells in the tissue wherein the nerves evoke potentials across the gap junctions which result in signals detected by the recording electrodes. Also, a method of stimulating peripheral nerves in the tissue with signals which do not activate the muscle cells and then monitoring the signals generated by the muscle cells from stimulating only the nerves, is disclosed.

Ash et al., U.S. Pat. No. 3,920,003, discloses an apparatus for detecting, amplification and identification of naturally generated low direct current potential differences in a mammalian body for the detection of ovulation, oestrous, menopause and pregnancy in mammals with a vaginal probe.

Matsuura, U.S. Pat. No. 5,109,865, discloses a method of diagnosing the types and stages of ovarian disorders or the presence of pregnancy from the results of measured sodium ion concentration in vaginal mucosa and change therein, based on the impedance value between electrodes on the vaginal mucosa.

### GENERAL DESCRIPTION OF THE INVENTION

According to a first aspect of the invention, there is provided a method of determining hormone activity in a mammal, the method including the step of measuring muscle tissue impedance of the mammal.

The hormone activity may be the reproductive or progesterone hormone activity of a female mammal.

In this specification, the phrase reproductive hormone or progesterone activity shall be understood to mean the level or concentration of reproductive hormones or progesterone hormone present in the body of a mammal.

The method may include the step of calibrating a muscle tissue impedance measuring apparatus such that an impedance measurement of the muscle tissue represents reproductive hormone activity.

The calibration may include taking a series of impedance readings of muscle tissue of a mammal over a period of time, to establish a substantially upper and lower limit of impedance readings corresponding to a substantially upper and lower limit, respectively, of reproductive hormone or progesterone cycle of the mammal.

Taking impedance readings and measurements may include the steps of generating a series of muscle stimulating electronic pulses either in the form of a fixed amplitude current pulse or in the form of a fixed amplitude voltage pulse, applying the pulses across muscle tissue and measuring the resulting voltage amplitude or current amplitude, as the case may be, allowing the impedance of the muscle tissue to be determined.

It is important that the method incorporates impedance measurements, which is frequency dependent, because of the capacitive nature of the muscle tissue. The pulse is selected such that the stimulation of the muscle tissue simulates natural body stimulation.

It is to be understood that the impedance of the muscles, which is dependent on hormonal activity effecting the ion transport mechanisms of the cells, effects the voltage amplitude of the applied pulses in the event of fixed amplitude current pulses and the current amplitude in the event of fixed amplitude voltage pulses.

It is further understood that the specific impedance of a muscle tissue may vary, from one individual mammal to another, also depending on the type of muscle tissue measured, and the method and location of taking the measurement.

In a preferred embodiment, a fixed current pulse, used for measurements and readings, may be selected between 80 mA and 240 mA corresponding to a voltage amplitude of between 1 and 3 volt, preferably between 1.2 and 2.6 volt, with a selected pulse frequency of approximately +/−50 Hz.

The voltage amplitude, current amplitude and/or the pulse frequency of an electronic pulse may be adjusted or calibrated to obtain an optimum sensitivity for a specific muscle tissue for each mammal depending on the upper and lower limits of impedance readings obtained for the specific muscle tissue during calibration.

The electronic pulse may be applied by a multi-polar probe electrode, which is inserted into the rectum. The pulses may be applied to the muscle wall of the intestines in the proximity of the womb.

In another embodiment of the invention, a series of electronic pulses used for measurements and readings may be applied to muscles over the length of the body by using clamp electrodes with the one pole clipped onto the mouth of a mammal and the other to the skin fold of its tail.

In a further embodiment of the invention, the electronic pulses may be applied by a set of needle electrodes inserted into the muscles of, for example, the inner tail or, on top at the base of the spine.

It will be appreciated that electronic pulses could also be applied to the vaginal tract of a mammal, however, the applicant believes this is not a suitable non-intrusive location for muscle impedance measurement and may lead to disadvantageous infections, and is therefore not recommended.

The livestock may include, but not be limited to, cattle, sheep, horses and pigs.

According to a second aspect of the invention, there is provided a method of monitoring the progress of a female mammal's reproductive hormone or progesterone cycle by determining reproductive hormone activity of the mammal, as described above, over an initial period of time.

It is sufficient to determine reproductive hormone activity for the time period between two rises of impedance readings over a time period, which corresponds to two drops in progesterone levels over the time period, representing a complete reproductive hormone or progesterone cycle of the mammal.

The method may include determining reproductive hormone or progesterone activity of the mammal at a later time and comparing the later hormone activity with the hormone activity of the complete reproductive hormone or progesterone cycle of the mammal determined over the initial period of time to determine at what stage of the reproductive hormone or progesterone cycle the mammal is at the time of measurement.

The method may also include determining a series of reproductive hormone activity of the mammal over a later period of time and
comparing the later hormone activity with the hormone activity of the complete progesterone cycle of the mammal to predict the cycle in the future and/or to determine whether the cycle of the mammal is normal and/or regular.

According to a third aspect of the invention, there is provided an apparatus for measuring, determining and/or monitoring reproductive hormone or progesterone activity as described above, the apparatus including:
an electronic pulse generator;
an electrode; and
a means for measuring impedance across the poles of the electrode.

The electrode may be in the form of a multi-polar probe electrode, clamps or needles.

The apparatus may also include a means to input an identifier, of saving measured data and of calculating a trend corresponding to the progesterone cycle in individual livestock.

The pulse generator may generate multiple fixed current pulses of between 80 mA and 240 mA corresponding to a voltage amplitude of between 1 to 3 volt, preferably between 1.2 and 2.6 volt.

The pulse generator may generate a series of electronic pulses at a frequency of +/−50 Hz, ranging from 30 Hz to 200 Hz.

An embodiment of the electrode may comprise an elongated rounded probe member of +/−20 mm in diameter with a typical length of 250 mm such that it may easily be inserted into the rectum of individual livestock for measurements across the intestine muscles. The distance of non conductive material between contacts of the electrode may be between 15 to 20 mm, typically with one pole at the tip and others down the length of the electrode.

Alternatively, the electrode may comprise a set of clamps, one clipped to the mouth and the other to the skin fold of the tail for measurements over the length of the back muscles.

A further embodiment of the electrode may comprise one or more sets of needles inserted into the muscles of the body including the base of the tail either on top or below and attached with adhesive tape for measurements across the muscles of the tail.

The means of measuring the impedance may use a peak voltage detector, which includes a sampling and holding mechanism, an averaging circuit and a level adjusting means.

The apparatus may include a portable power source such as a battery.

The apparatus may further include a display for displaying the calibrated measurements.

The apparatus may be mobile, preferably portable.

The apparatus may further include a memory for storing readings and a processor for calibration and control of measurements.

According to a fourth aspect of the invention, there is provided a system for monitoring the reproductive hormone or progesterone activity of livestock, the system including:
an apparatus for determining or measuring the reproductive hormone or progesterone activity of an individual of the livestock, as described above; and
a database comprising the determined or measured hormone activity of each individual of the livestock over a period of time.

The system may include a means for allocating an identifying code to each individual of livestock. The code may be used to identify an individual animal and for allocation of that animal's hormone activity readings or measurements stored in a memory of the apparatus. The code may also be used to allocate the hormone activity data of an individual animal in the database.

The system may include a means for comparing a measurement of reproductive hormone or progesterone activity of an animal with its data, such as calibration data, on the database to determine the stage of the reproductive hormone or progesterone cycle of the animal.

The system may be in the form of a digital computer network. The database may be kept on a central server, personal computer, notebook or hand held computer. The apparatus for determining or measuring the hormone activity of an individual of the livestock may be mobile and may be provided with a memory for storing measurements. The network may be wireless and the apparatus may include an interface or transmitter for network communication with the central server, personal computer, notebook computer or hand held computer. Instead, the entire database may be stored in the memory of the apparatus.

The invention also extends to a method, apparatus and system to determine whether an animal is pregnant, by means of monitoring or determining reproductive hormone or progesterone activity of the mammal as described above.

This invention extends further to determining factors, other than reproductive hormone activity, which factors effects the electrochemical properties of mammalian cells, such as for example health, stress, pain etc. These factors may, for example, cause a hormonal reaction in the mammal, which, in turn, effects the electrochemical properties of its cells. The electrochemical properties can be measured or determined with an apparatus and method in accordance with the invention.

### DETAILED DESCRIPTION OF THE INVENTION

The invention is now described, by way of non-limiting examples, with reference to the accompanying diagrammatic drawings.

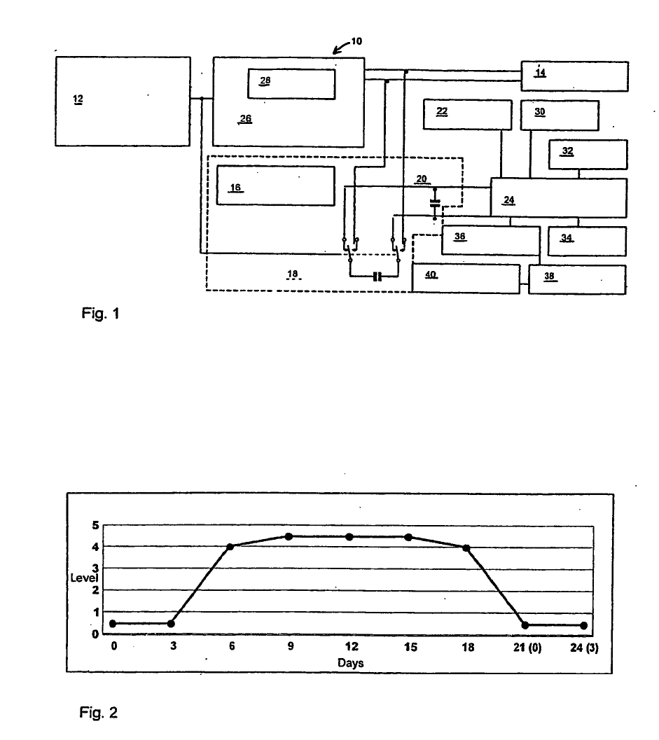

FIG. 1 shows diagrammatically an apparatus for measuring, determining and/or monitoring reproductive hormone or progesterone activity, in accordance with the invention; and

FIG. 2 shows a graph of reproductive hormone or progesterone hormone activity of a cow over a period of 24 days.

A portable apparatus for measuring, determining and/or monitoring reproductive hormone or progesterone activity of a female mammal of livestock is generally indicated by reference numeral 10. The apparatus 10, includes an electronic current pulse generator 12, a multi-polar electrode 14 and a means for measuring impedance across the poles of the electrode 14, in the form of a peak voltage detector 16, which includes a sampling 18 and holding 20 capacitor mechanism for obtaining an average direct current reading. The peak detector 16 also includes an averaging circuit and level adjusting means (not shown). The apparatus 10 further includes a memory 22 for storing measured data and a processor 24 for calculating a trend corresponding to the progesterone cycle in individual livestock or comparing a measurement of reproductive hormone or progesterone activity of an animal with its data on the database to determine the stage of the reproductive hormone or progesterone cycle of the animal.

The pulse generator 12 generates multiple current pulses of between 80 mA and 240 mA corresponding to a voltage amplitude of between 1.2 and 2.6 volt and a current amplitude pulses at a frequency of +/−50 Hz.

The electrode 14 comprises a rounded probe member (not shown) 20 mm in diameter with a length of 250 mm such that it may easily be inserted into the rectum of individual livestock. The distance between contacts of the electrode 14 is 15 mm, but in other embodiments of the invention it can range from approximately 10 mm to 20 mm. The apparatus also includes a constant current source 26 powered by a battery 28. The apparatus further includes a display 30 for displaying the calibrated or measured voltage amplitude.

The apparatus 10 for measuring, determining and/or monitoring reproductive hormone or progesterone activity is of a digital design with the display 30 being in the form of a digital read-out. The apparatus 10 is further provided with an alphanumeric keypad 32 for input of the livestock identification number. Alternatively, a sensor 34 may automatically pick up an electronic identification tag attached to the animal. The apparatus 10 is further provided with an interface 36 for downloading measurement data to a computer 38.

In another embodiment of invention, the apparatus 10 may be a miniaturised unit attached to the tail or base of the spine, the apparatus taking intermittent measurements and transmitting the measurement data at regular intervals to a base station 40 for downloading onto a computer 38.

In use, the apparatus 10 for measuring, determining and/or monitoring reproductive hormone or progesterone activity is calibrated such that an impedance measurement of the muscle tissue represents reproductive hormone or progesterone activity. The calibration includes taking a series of impedance readings of muscle tissue of an animal over a period of time, to establish a substantially upper and lower limit of impedance readings, corresponding to a substantially upper and lower limit, respectively, of reproductive hormone or progesterone activity of a progesterone cycle of the animal, as shown in FIG. 2. The calibration data is allocated to the specific animal and stored in the memory 22 of the apparatus 10. In order to determine the reproductive hormone activity of the animal at any time the muscle tissue impedance is measured and compared with the calibration data.

The apparatus 10 is conveniently configured to store simultaneously, the calibration data of different types of livestock.

Normally a total of 8 to 10 measurements with an interval of 2 to 4 days between measurements over a period of approximately 26 days in cattle, for example, are required to calibrate the apparatus 10 and to determine the reproductive hormone or progesterone cycle. Alternatively, after establishing the progesterone cycle, a number of measurements may be made on the day ovulation is expected, to determine within 1 or 2 hours the moment of the occurrence of ovulation, to assist in the timing of the artificial insemination procedure.

In the event that the reproductive hormone or progesterone cycle shows irregularities in the absence of impregnation or artificial insemination, it may be deduced that an ovarian disorder is present. A complete measurement cycle should be completed before attempting artificial insemination or allowing the female to mate in order to determine that the female has a healthy reproductive system.

Normally, the reproductive hormone or progesterone cycle repeats the same pattern in the absence of conception as shown in FIG. 2. Day 0 in the Cycle is taken as when an egg cell is released by the follicle.

After approximately 5 to 7 days the Corpus Luteum develops which releases high levels of progesterone until day 16 to 18, when it is destroyed. Typically on day 21 a new follicle is formed and the cycle repeats.

In the event of conception, the pattern is disrupted in preparation of the womb to carry the foetus. Once the egg is fertilised a protein is released which prevents the Corpus Luteum from being destroyed. The disruption can therefore be used to indicate pregnancy, as the cycle will not repeat during pregnancy. In the event that the cycle recommences after an interval of time it may be deduced that the foetus has been aborted.

It shall be understood that the examples are provided for illustrating the invention further and to assist a person skilled in the art with understanding the invention and are not meant to be construed as unduly limiting the reasonable scope of the invention.

1. A method of determining hormone activity in a mammal, the method including the steps of measuring muscle tissue impedance of the mammal and calibrating a muscle tissue impedance measuring apparatus such that an impedance measurement of the muscle tissue represents hormone activity.

2. A method as claimed in claim 1, wherein the hormone activity is the reproductive hormone activity of a female mammal.

3-44. (CANCELED)

45. A method as claimed in claim 2, wherein the calibration step includes taking a series of impedance readings of muscle tissue of the female mammal over a period of time, to establish a substantially upper and lower limit of impedance readings corresponding to a substantially upper and lower limit, respectively, of reproductive hormone or progesterone cycle of the female mammal.

46. A method as claimed in claim 45, wherein the step of measuring muscle tissue impedance includes the steps of generating a series of muscle stimulating electronic pulses in the form of a fixed amplitude current pulse, applying the pulses across muscle tissue and measuring the resulting voltage amplitude, allowing the impedance of the muscle tissue to be determined.

47. A method as claimed in claim 45, wherein the step of measuring muscle tissue impedance includes the steps of generating a series of muscle stimulating electronic pulses in the form of a fixed amplitude voltage pulse, applying the pulses across muscle tissue and measuring the resulting current amplitude, allowing the impedance of the muscle tissue to be determined.

48. A method as claimed in claim 46, wherein each electronic pulse is a fixed current pulse selected to be between 80 mA and 240 mA corresponding to a voltage amplitude of between 1 and 3 volt with a selected pulse frequency of approximately 50 Hz.

49. A method as claimed in claim 48, wherein the voltage amplitude is between 1.2 and 2.6 volt.

50. A method as claimed in claim 1, wherein the hormone activity is hormone activity related to factors, other than reproductive hormone activity, which factors effects the electrochemical properties of mammalian cells.

51. An apparatus for measuring, determining and/or monitoring hormone activity of a mammal by measuring muscle tissue impedance of the mammal, the apparatus including:
an electronic pulse generator;
an electrode;
a means for measuring impedance across the poles of the electrode;
a means of saving measured data;
a means of calculating a trend in the saved measured data corresponding to hormone activity in individual mammal.

52. An apparatus as claimed in claim 51, wherein the hormone activity is reproductive hormone activity.

53. An apparatus as claimed in claim 52, wherein the electrode is in the form selected from a multi-polar probe electrode, clamps and needles.

54. An apparatus as claimed in claim 52, wherein the pulse generator generates multiple fixed current pulses of between 80 mA and 240 mA corresponding to a voltage amplitude of between 1 to 3 volt.

55. An apparatus as claimed in claim 54, wherein the voltage amplitude is between 1.2 and 2.6 volt.

56. An apparatus as claimed in claim 55, wherein the pulse generator generates a series of electronic pulses at a frequency ranging from 30 Hz to 200 Hz.

57. An apparatus as claimed in claim 56, wherein the frequency is about 50 Hz.

58. An apparatus as claimed in claim 56, wherein the means of measuring the impedance includes a peak voltage detector, which includes a sampling and holding mechanism, an averaging circuit and a level adjusting means.

59. A system for monitoring the hormone activity of livestock, the system including:
an apparatus for determining or measuring the hormone activity of an individual of the livestock as claimed in claim 1;
a database comprising the determined or measured hormone activity of each individual of the livestock over a period of time; and
a means for calibrating a muscle tissue impedance measuring apparatus such that an impedance measurement of the muscle tissue represents hormone activity.

60. A system as claimed in claim 59, which includes an allocating means for allocating an identifying code to each individual of livestock.

61. A system as claimed in claim 59, wherein the hormone activity is reproductive hormone activity.

62. A system as claimed in claim 59, which includes a comparison means for comparing a measurement of reproductive hormone activity of a mammal with its data, such as calibration data, on the database to determine the stage of the reproductive hormone or progesterone cycle of the animal.
